(19) United States
(12) Patent Application Publication
(10) Pub. No.: US 2022/0378368 A1
(43) Pub. Date: Dec. 1, 2022

(54) PAIN ESTIMATION APPARATUS, PAIN ESTIMATION METHOD, AND RECORDING MEDIUM

(71) Applicant: OLYMPUS CORPORATION, Tokyo (JP)

(72) Inventors: Koichi TAKAYAMA, Tokyo (JP); Hiromasa EUJITA, Tokyo (JP)

(73) Assignee: OLYMPUS CORPORATION, Tokyo (JP)

(21) Appl. No.: 17/884,813

(22) Filed: Aug. 10, 2022

Related U.S. Application Data
(63) Continuation of application No. PCT/JP2020/005815, filed on Feb. 14, 2020.

(51) Int. Cl. A61B 5/00; A61B 1/005

(52) U.S. Cl. CPC A61B 5/4824; A61B 1/009; A61B 2560/0266

(57) ABSTRACT

A pain estimation apparatus includes: an information acquisition unit configured to perform a process for acquiring, in an endoscopic examination, examination state information for estimation including insertion state information for estimation including at least one of insertion shape information for estimation about an insertion shape of an insertion section of an endoscope inserted inside a body of a subject or operation force amount information for estimation about an amount of force applied to the insertion section, and information different from either of the insertion shape information for estimation and the operation force amount information for estimation; and a pain estimation processing means configured to generate pain information about pain of the subject based on the examination state information for estimation including the information different from either of the insertion shape information for estimation and the operation force amount information for estimation, and the insertion state information for estimation.

### CROSS REFERENCE TO RELATED APPLICATION

This application is a continuation application of PCT/JP2020/005815 filed on Feb. 14, 2020, the entire contents of which are incorporated herein by this reference.

### BACKGROUND OF THE INVENTION

### 1. Field of the Invention

The present invention relates to a pain estimation apparatus, a pain estimation method, and a recording medium that are used at the time of an endoscopic examination.

### 2. Description of the Related Art

In an endoscopic examination in a medical field, an insertion operation of inserting an elongated insertion section provided on an endoscope deep inside a body of a subject such as a patient is performed. Furthermore, in relation to the endoscopic examination in the medical field, proposals have conventionally been made regarding acquisition of information for supporting the insertion operation of the insertion section of the endoscope.

More specifically, for example, International Publication No. 2018/135018 discloses a method for calculating, as information for supporting the insertion operation of the insertion section of the endoscope, a force that is applied to the insertion section inserted inside a body of a subject.

In relation to the endoscopic examination in the medical field, a method for estimating a degree of pain of a subject into whom the insertion section of the endoscope is inserted (who is taking the endoscopic examination) is being studied.

### SUMMARY OF THE INVENTION

A pain estimation apparatus according to a mode of the present invention includes: an information acquisition unit configured to perform a process for acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and pain estimation processing means configured to generate pain information about pain of the subject based on the examination state information for estimation including the information different from either of the insertion shape information for estimation and the operation force amount information for estimation, and the insertion state information for estimation.

A pain estimation method according to a mode of the present invention includes: acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and generating pain information about pain of the subject based on the examination state information for estimation including the information different from either of the insertion shape information for estimation and the operation force amount information for estimation, and the insertion state information for estimation.

A non-transitory recording medium according to a mode of the present invention records a program. The program causes a computer to: perform a process for acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and perform a process for generating pain information about pain of the subject based on the examination state information for estimation including the information different from either of the insertion shape information for estimation and the operation force amount information for estimation, and the insertion state information for estimation.

### BRIEF DESCRIPTION OF THE DRAWINGS

FIG. 1 is a diagram showing a configuration of main parts of an endoscope system including a pain estimation apparatus according to an embodiment;

FIG. 2 is a block diagram for describing a specific configuration of the endoscope system according to the embodiment; and

FIG. 3 is a schematic diagram showing an example of an estimation model used in processing by the pain estimation apparatus according to the embodiment.

### DETAILED DESCRIPTION OF THE PREFERRED EMBODIMENTS

Hereinafter, an embodiment of the present invention will be described with reference to the drawings.

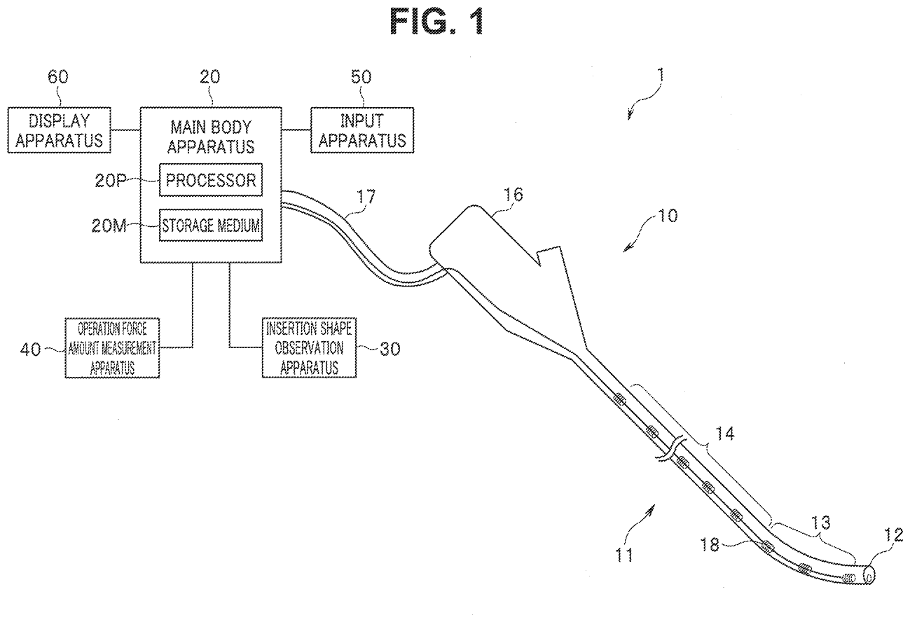

As shown in FIG. 1, an endoscope system 1 includes an endoscope 10, a main body apparatus 20, an insertion shape observation apparatus 30, an operation force amount measurement apparatus 40, an input apparatus 50, and a display apparatus 60, for example. FIG. 1 is a diagram showing a configuration of main parts of an endoscope system including a pain estimation apparatus according to the embodiment.

The endoscope 10 includes an insertion section 11 to be inserted into a body of a subject such as a patient, an operation section 16 provided on a proximal end side of the insertion section 11, and a universal cord 17 extending from the operation section 16. The endoscope 10 is removably connected to the main body apparatus 20 via a scope connector (not shown) provided at an end portion of the universal cord 17. Moreover, a light guide 110 (not shown in FIG. 1) for transmitting illumination light supplied by the main body apparatus 20 is provided inside the insertion section 11, the operation section 16, and the universal cord 17.

The insertion section 11 has a flexible, elongated shape. The insertion section 11 includes, in the stated order from a distal end side, a distal end portion 12 that is rigid, a bending portion 13 that is formed bendable, and a flexible tube portion 14 that is long and that has flexibility. Moreover, a plurality of source coils 18 that generate a magnetic field according to a coil drive signal supplied from the main body apparatus 20 are provided inside the distal end portion 12, the bending portion 13, and the flexible tube portion 14. The plurality of source coils 18 are disposed at predetermined intervals in a longitudinal direction of the insertion section 11. A gas-feeding channel 120 (not shown in FIG. 1) that is formed as a conduit for allowing gas supplied from the main body apparatus 20 to flow through and to be discharged forward from the distal end portion 12 is provided inside the insertion section 11. A rigidity changing mechanism 130 (not shown in FIG. 1) is provided inside a variable rigidity range provided in at least a partial range of the insertion section 11, in the longitudinal direction of the insertion section 11. The rigidity changing mechanism 130 is configured to be capable of changing flexural rigidity of the variable rigidity range by being controlled by the main body apparatus 20. Note that, in the following, “flexural rigidity” will be abbreviated as appropriate as “rigidity” for the sake of description.

An illumination window (not shown) for emitting, toward an object, illumination light transmitted by the light guide 110 provided inside the insertion section 11 is provided in the distal end portion 12. An image pickup unit 140 (not shown in FIG. 1) is also provided in the distal end portion 12. The image pickup unit 140 is configured to perform operation according to an image pickup control signal supplied from the main body apparatus 20, and to perform image pickup of an object illuminated by the illumination light emitted through the illumination window and output a picked-up image signal.

The bending portion 13 is configured to be bendable according to an operation of an angle knob (not shown) provided on the operation section 16.

The operation section 16 has a shape that allows a user to grasp and operate. The operation section 16 is provided with the angle knob that is configured to allow an operation of causing the bending portion 13 to bend in four directions of up, down, left and right that intersect a longitudinal axis of the insertion section 11. The operation section 16 is also provided with at least one scope switch (not shown) that is capable of carrying out an instruction according to an input operation by the user.

The main body apparatus 20 includes at least one processor 20P, and a non-transitory storage medium 20M. The main body apparatus 20 is removably connected to the endoscope 10 via the universal cord 17. The main body apparatus 20 is also removably connected to each of the insertion shape observation apparatus 30, the input apparatus 50, and the display apparatus 60. The main body apparatus 20 is configured to perform an operation according to an instruction from the input apparatus 50. The main body apparatus 20 is configured to generate an endoscopic image based on the picked-up image signal outputted from the endoscope 10, and to perform an operation of causing the display apparatus 60 to display the endoscopic image that is generated. The main body apparatus 20 is also configured to generate and output various control signals for controlling operation of the endoscope 10. The main body apparatus 20 includes a function of the pain estimation apparatus, and is configured to estimate a degree of pain of a subject taking an endoscopic examination and acquire an estimation result, and to perform a process for generating pain level information indicating the estimation result that is acquired. The main body apparatus 20 is further configured to be capable of performing an operation of causing the display apparatus 60 to display the pain level information generated in the above manner.

The insertion shape observation apparatus 30 is configured to detect a magnetic field generated by each source coil 18 provided in the insertion section 11, and to acquire a position of each of the plurality of source coils 18 based on intensity of the magnetic field that is detected. The insertion shape observation apparatus 30 is further configured to generate, and output to the main body apparatus 20, insertion position information indicating the position of each of the plurality of source coils 18 acquired in the above manner.

For example, the operation force amount measurement apparatus 40 includes a myoelectric sensor that is capable of measuring muscle potential generated in a hand or an arm of the user operating the endoscope 10. The operation force amount measurement apparatus 40 is configured to measure a value of voltage generated according to an operation force amount applied to the insertion section 11 by the user operating the endoscope 10, and to generate, and output to the main body apparatus 20, operation force amount information indicating the measured value of the voltage.

Note that, in the present embodiment, the operation force amount measurement apparatus 40 may be configured to acquire a measurement result obtained by measuring a value or the like of voltage generated according to an operation force amount applied to the insertion section 11 by a robot, not shown, that is capable of operating the endoscope 10, and to generate, and output to the main body apparatus 20, operation force amount information indicating the measurement result that is acquired.

For example, the input apparatus 50 includes at least one input interface to be operated by the user, such as a mouse, a keyboard or a touch panel. The input apparatus 50 is configured to be capable of outputting information and instructions inputted according to operation by the user, to the main body apparatus 20.

For example, the display apparatus 60 includes a liquid crystal monitor. The display apparatus 60 is configured to be capable of displaying, on a screen, endoscopic images and the like outputted from the main body apparatus 20.

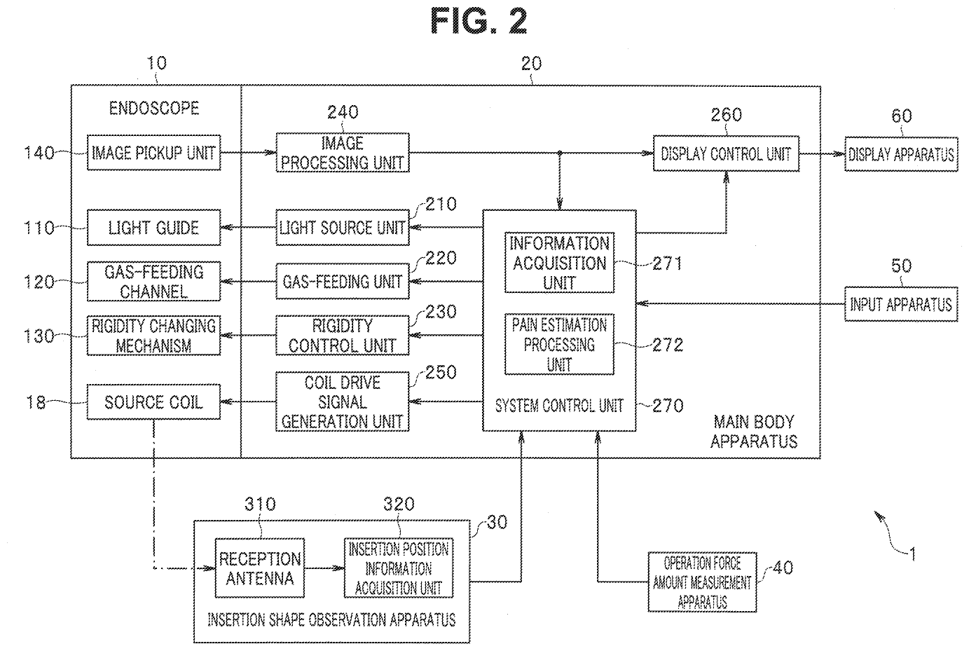

As shown in FIG. 2, the endoscope 10 includes the source coils 18, the light guide 110, the gas-feeding channel 120, the rigidity changing mechanism 130, and the image pickup unit 140. FIG. 2 is a block diagram for describing a specific configuration of the endoscope system according to the embodiment.

For example, the image pickup unit 140 includes an observation window through which return light from an object illuminated by the illumination light enters, and an image sensor, such as a color CCD, that images the return light and outputs a picked-up image signal.

As shown in FIG. 2, the main body apparatus 20 includes a light source unit 210, a gas-feeding unit 220, a rigidity control unit 230, an image processing unit 240, a coil drive signal generation unit 250, a display control unit 260, and a system control unit 270.

For example, the light source unit 210 includes, as a light source, at least one LED or at least one lamp. The light source unit 210 is configured to be capable of generating illumination light for illuminating an inside of a body of a subject into whom the insertion section 11 is inserted, and of supplying the illumination light to the endoscope 10. The light source unit 210 is also configured to be capable of changing an amount of light of the illumination light according to a system control signal supplied from the system control unit 270.

For example, the gas-feeding unit 220 includes a pump or a tank for feeding gas. The gas-feeding unit 220 is configured to perform an operation of supplying gas stored in the tank to the gas-feeding channel 120, according to a system control signal supplied from the system control unit 270.

For example, the rigidity control unit 230 includes a rigidity control circuit. The rigidity control unit 230 is configured to perform an operation of setting a degree of rigidity in the variable rigidity range of the insertion section 11 by controlling a drive state of the rigidity changing mechanism 130 according to a system control signal supplied from the system control unit 270.

For example, the image processing unit 240 includes an image processing circuit. The image processing unit 240 is configured to generate an endoscopic image by performing a predetermined process on a picked-up image signal outputted from the endoscope 10, and to output the endoscopic image that is generated to the display control unit 260 and the system control unit 270.

For example, the coil drive signal generation unit 250 includes a drive circuit. The coil drive signal generation unit 250 is configured to generate and output a coil drive signal for driving the source coil 18, according to a system control signal supplied from the system control unit 270.

The display control unit 260 is configured to perform a process for generating a display image including the endoscopic image outputted from the image processing unit 240, and to perform a process for causing the display apparatus 60 to display the display image that is generated. The display control unit 260 is also configured to perform a process for causing the display apparatus 60 to display the pain level information outputted from the system control unit 270. Various types of information, including the pain level information, displayed by the display apparatus 60 are communicated to a doctor, who is a user, or a nurse or the like, who is a healthcare worker other than the user.

The system control unit 270 is configured to generate and output system control signals for causing operations to be performed according to instructions and the like from the operation section 16 and the input apparatus 50. The system control unit 270 includes an information acquisition unit 271, and a pain estimation processing unit 272.

The information acquisition unit 271 is configured to perform a process for acquiring insertion state information (insertion state information for estimation) corresponding to information indicating an insertion state of the insertion section 11 inserted inside a body of a subject, based on the insertion position information outputted from the insertion shape observation apparatus 30.

More specifically, for example, the information acquisition unit 271 is configured to calculate a plurality of curvatures at respective positions of the plurality of source coils 18 provided in the insertion section 11, based on a plurality of three-dimensional coordinate values (described later) included in the insertion position information outputted from the insertion shape observation apparatus 30 and acquire a calculation result, and to generate insertion state information including the calculation result that is acquired. In other words, the information acquisition unit 271 is configured to perform a process of calculating a plurality of curvatures at a plurality of positions, respectively, in the insertion section 11, as a process for obtaining information (insertion shape information for estimation) about an insertion shape of the insertion section 11 that is inserted inside a body of a subject taking one endoscopic examination.

The pain estimation processing unit 272 is configured to perform a process for acquiring the estimation result of estimating a degree of pain of the subject, based on the insertion state information generated by the information acquisition unit 271. The estimation result of the degree of pain of the subject is acquired as one pain level among a predetermined plurality of pain levels, for example. The pain estimation processing unit 272 is configured to generate the pain level information (pain information) indicating the estimation result acquired in the above manner, and to output the pain level information that is generated to the display control unit 260.

A specific example of a configuration of the pain estimation processing unit 272 according to the present embodiment will be described.

The pain estimation processing unit 272 is configured to obtain the estimation result that estimates one pain level among the predetermined plurality of pain levels to be the degree of pain of the subject corresponding to the insertion state information generated by the information acquisition unit 271, by performing processing using an estimator CLP that is created through learning, by a learning method such as deep learning, of each connection coefficient (weight) of a multi-layer neural network including an input layer, a hidden layer, and an output layer.

At the time of creating the estimator CLP mentioned above, for example, machine learning is performed using training data including insertion state information similar to the insertion state information generated by the information acquisition unit 271 and a label indicating a classification result of classifying the degree of pain of a subject corresponding to the insertion state information into one pain level among the predetermined plurality of pain levels. In other words, the estimator CLP is created by performing machine learning using, as the training data, pre-collected information including insertion shape information for pre-collection that is a pre-collection target, and pain information for pre-collection indicating the degree of pain of a subject corresponding to the insertion shape information for pre-collection, for example. Furthermore, each of the predetermined plurality of pain levels is set as a level among multiple stages including great, small and zero, for example. Furthermore, at the time of creating the training data as mentioned above, for example, a task is performed, by which a label according to an evaluation result of evaluating the degree of pain based on a subjective evaluation criterion of a subject taking an endoscopic examination, such as a pushed state of a push button switch including a plurality of switches to be pushed by the subject according to the degree of pain actually occurring in the subject, is assigned to the insertion state information. Alternatively, at the time of creating the training data as mentioned above, a task is performed, by which a label according to an evaluation result of evaluating the degree of pain based on an objective evaluation criterion provided by a person other than the subject, such as an expert, is assigned to the insertion state information. The objective evaluation criterion uses a result of analysis of a waveform obtained by an electroencephalograph that measures a brain wave of a subject taking an endoscopic examination, a result of analysis of a waveform obtained by the myoelectric sensor that measures muscle potential generated in the subject, and the like.

Accordingly, with the estimator CLP mentioned above, by inputting, as input data, a plurality of curvatures included in the insertion state information generated by the information acquisition unit 271 to the input layer of the neural network, a plurality of likelihoods corresponding to respective levels that may be estimated to be the pain level of the subject corresponding to the insertion state information may be acquired as output data outputted from the output layer of the neural network, for example. Furthermore, with processing using the estimator CLP mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the insertion state information, generated by the information acquisition unit 271 in one endoscopic examination, to the estimator CLP corresponding to an estimation model created by machine learning using information similar to the insertion state information, obtained prior to the one endoscopic examination, the pain estimation processing unit 272 obtains the estimation result estimating one pain level, among the predetermined plurality of pain levels, to be the degree of pain of the subject in the one endoscopic examination.

In the present embodiment, functions of the main body apparatus 20 may at least be partially implemented by the processor 20P. In the present embodiment, at least a part of the main body apparatus 20 may be a separate electronic circuit, or a circuit block of an integrated circuit such as an FPGA (field programmable gate array). By modifying the components of the present embodiment as appropriate, a computer may be made to read a program for performing at least a part of the functions of the main body apparatus 20 from the storage medium 20M, such as a memory, and to perform an operation according to the program that is read.

As shown in FIG. 2, the insertion shape observation apparatus 30 includes a reception antenna 310, and an insertion position information acquisition unit 320.

For example, the reception antenna 310 includes a plurality of coils for three-dimensionally detecting the magnetic field generated by each of the plurality of source coils 18. The reception antenna 310 is configured to detect the magnetic field generated by each of the plurality of source coils 18, and to generate, and output to the insertion position information acquisition unit 320, a magnetic field detection signal according to the intensity of the magnetic field that is detected.

The insertion position information acquisition unit 320 is configured to acquire the position of each of the plurality of source coils 18 based on the magnetic field detection signal outputted from the reception antenna 310. The insertion position information acquisition unit 320 is configured to generate, and output to the system control unit 270, the insertion position information indicating the position of each of the plurality of source coils 18 acquired in the above manner

More specifically, the insertion position information acquisition unit 320 acquires, as respective positions of the plurality of source coils 18, a plurality of three-dimensional coordinate values in a spatial coordinate system that is virtually set by taking, as an origin or a reference point, a predetermined position (such as anus) on the subject into whom the insertion section 11 is inserted, for example. The insertion position information acquisition unit 320 generates, and outputs to the system control unit 270, the insertion position information including the plurality of three-dimensional coordinate values that are acquired in the above manner

In the present embodiment, at least a part of the insertion shape observation apparatus 30 may be an electronic circuit, or a circuit block of an integrated circuit such as an FPGA (field programmable gate array). In the present embodiment, the insertion shape observation apparatus 30 may include one or more processors (such as CPU), for example.

Next, effects of the present embodiment will be described.

A user, such as a surgeon, connects each part of the endoscope system 1 and turns on power, and then, disposes the insertion section 11 such that the distal end portion 12 is positioned near the anus or rectum of the subject, for example.

The information acquisition unit 271 performs a process for generating the insertion state information including the calculation result of the plurality of curvatures at the respective positions of the plurality of source coils 18 provided in the insertion section 11, based on the insertion position information outputted from the insertion shape observation apparatus 30.

The pain estimation processing unit 272 acquires the estimation result of the pain level of the subject according to the insertion state information generated by the information acquisition unit 271, by inputting the plurality of curvatures included in the insertion state information to the estimator CLP and performing processing, and generates the pain level information indicating the estimation result that is acquired. Then, the pain estimation processing unit 272 outputs the pain level information that is generated in the above manner, to the display control unit 260.

More specifically, the pain estimation processing unit 272 acquires the estimation result estimating that the degree of pain of the subject is at one pain level among a pain level PH corresponding to a case where the pain occurring in the subject is relatively great, a pain level PL corresponding to a case where the pain occurring in the subject is relatively small, and a pain level PN corresponding to a case where there is no occurrence of pain in the subject, for example.

The display control unit 260 performs a process for causing the display apparatus 60 to display the pain level information outputted from the pain estimation processing unit 272.

More specifically, for example, in the case where the pain level indicated by the pain level information outputted from the pain estimation processing unit 272 is PH, the display control unit 260 generates text indicating that the pain occurring in the subject is great, and performs a process for causing the display apparatus 60 to display the text that is generated. For example, in the case where the pain level indicated by the pain level information outputted from the pain estimation processing unit 272 is PL, the display control unit 260 generates text indicating that the pain occurring in the subject is small, and performs a process for causing the display apparatus 60 to display the text that is generated. For example, in the case where the pain level indicated by the pain level information outputted from the pain estimation processing unit 272 is PN, the display control unit 260 generates text indicating that there is no occurrence of pain in the subject, and performs a process for causing the display apparatus 60 to display the text that is generated.

As described above, according to the present embodiment, the degree of pain occurring in a subject taking an endoscopic examination may be estimated, and information indicating the degree of pain of the subject may be presented to the user. Therefore, according to the present embodiment, a burden on the user who performs an insertion operation of the insertion section of the endoscope may be reduced.

According to the present embodiment, for example, the information acquisition unit 271 may generate an insertion shape image that two-dimensionally shows the insertion shape of the insertion section 11 that is inserted in the subject taking the endoscopic examination, based on the plurality of three-dimensional coordinate values included in the insertion position information outputted from the insertion shape observation apparatus 30, and perform a process for generating the insertion state information including the insertion shape image that is generated. In other words, in the present embodiment, the information acquisition unit 271 may be configured to perform a process of generating the insertion shape image that two-dimensionally shows the insertion shape of the insertion section 11, as the process for obtaining information about the insertion shape of the insertion section 11.

In the case as described above, the pain estimation processing unit 272 may be configured to obtain the estimation result that estimates one pain level among the predetermined plurality of pain levels to be the degree of pain of the subject corresponding to the insertion state information generated by the information acquisition unit 271, by performing processing using an estimator CLQ that is created through learning, by a learning method such as deep learning, of each connection coefficient (weight) of a multi-layer neural network including an input layer, a hidden layer, and an output layer.

At the time of creating the estimator CLQ mentioned above, for example, machine learning is performed using training data including insertion state information similar to the insertion state information generated by the information acquisition unit 271 and a label indicating a classification result of classifying the degree of pain of a subject corresponding to the insertion state information into one pain level among the predetermined plurality of pain levels. Furthermore, each of the predetermined plurality of pain levels is set as a level among multiple stages including great, small and zero, for example. Moreover, at the time of creating the training data as mentioned above, a task of assigning, to the insertion state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLQ mentioned above, by acquiring multi-dimensional data such as pixel values of respective pixels of the insertion shape image included in the insertion state information generated by the information acquisition unit 271, and inputting the multi-dimensional data to the input layer of the neural network as input data, a plurality of likelihoods corresponding to respective levels that may be estimated to be the pain level of the subject corresponding to the insertion state information may be acquired as output data outputted from the output layer of the neural network, for example. Furthermore, with processing using the estimator CLQ mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the insertion state information, generated by the information acquisition unit 271 in one endoscopic examination, to the estimator CLQ corresponding to an estimation model created by machine learning using information similar to the insertion state information, obtained prior to the one endoscopic examination, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may acquire, as the insertion state information, operation force amount information for estimation that is information about the amount of force that is applied to the insertion section 11 in an endoscopic examination. For example, time series data including a plurality of voltage values may be acquired as the operation force amount information for estimation, by recording, for a predetermined time period, one voltage value outputted from the operation force amount measurement apparatus 40, and a process for acquiring the insertion state information including the time series data that is acquired may be performed.

In the case as described above, the pain estimation processing unit 272 may be configured to obtain the estimation result that estimates one pain level among the predetermined plurality of pain levels to be the degree of pain of the subject corresponding to the insertion state information generated by the information acquisition unit 271, by performing processing using an estimator CLW that is created through learning, by a learning method such as deep learning, of each connection coefficient (weight) of a multi-layer neural network including an input layer, a hidden layer, and an output layer.

At the time of creating the estimator CLW mentioned above, for example, machine learning is performed using training data including insertion state information similar to the insertion state information generated by the information acquisition unit 271 and a label indicating a classification result of classifying the degree of pain of a subject corresponding to the insertion state information into one pain level among the predetermined plurality of pain levels. In other words, the estimator CLW is created by performing machine learning using, as the training data, pre-collected information including operation force amount information for pre-collection that is a pre-collection target, and the pain information for pre-collection indicating the degree of pain of a subject corresponding to the operation force amount information for pre-collection, for example. Furthermore, each of the predetermined plurality of pain levels is set as a level among multiple stages including great, small and zero, for example. Moreover, at the time of creating the training data as mentioned above, a task of assigning, to the insertion state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLW mentioned above, by inputting, as input data, a plurality of voltage values included in the time series data in the insertion state information generated by the information acquisition unit 271 to the input layer of the neural network, a plurality of likelihoods corresponding to respective levels that may be estimated to be the pain level of the subject corresponding to the insertion state information may be acquired as output data outputted from the output layer of the neural network, for example. Furthermore, with processing using the estimator CLW mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the insertion state information, generated by the information acquisition unit 271 in one endoscopic examination, to the estimator CLW corresponding to an estimation model created by machine learning using information similar to the insertion state information, obtained prior to the one endoscopic examination, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for acquiring, as information indicating an examination state of one endoscopic examination, examination state information (examination state information for estimation) including the insertion state information generated based on a plurality of three-dimensional coordinate values included in the insertion position information outputted from the insertion shape observation apparatus 30, and subject information (subject information for estimation) corresponding to information about the subject obtained by detecting information inputted to the input apparatus 50. Note that the subject information includes at least one piece of information among information indicating sex of the subject taking the endoscopic examination, information indicating age of the subject, information indicating body shape of the subject, information indicating presence/absence of intestinal tract adhesion in the subject, and information indicating use/non-use of a sedative by the subject, for example.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLR that is created through learning, by a learning method such as deep learning, of each connection coefficient (weight) of a multi-layer neural network including an input layer, a hidden layer, and an output layer.

At the time of creating the estimator CLR mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLR is created by performing machine learning using, as the training data, information including a relationship between the subject information for pre-collection and the pain information for pre-collection, and pre-collected information including a relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLR mentioned above, by inputting, as input data, a plurality of curvatures included in the insertion state information in the examination state information generated by the information acquisition unit 271 and a value corresponding to the subject information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLR mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLR that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for acquiring, as information indicating the examination state of one endoscopic examination, examination state information including the insertion state information generated based on a plurality of three-dimensional coordinate values included in the insertion position information outputted from the insertion shape observation apparatus 30 and endoscopic image information for estimation about an endoscopic image outputted from the image processing unit 240. For example, the endoscopic image information for estimation may be analysis information indicating an analysis result obtained by performing analysis processing on an endoscopic image outputted from the image processing unit 240. Note that it suffices if the analysis information mentioned above includes one of information indicating occurrence/non-occurrence of an excessive proximity state corresponding to a state where a distance from an intestinal wall of the subject taking the endoscopic examination (into whom the insertion section 11 is inserted) to a distal end surface of the distal end portion 12 is zero or substantially zero, and information indicating presence/absence of a diverticulum of an intestinal tract of the subject, for example. Occurrence/non-occurrence of the excessive proximity state may be detected based on a proportion of a red region in the entire endoscopic image outputted from the image processing unit 240, for example.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLS that is created through learning, by a learning method such as deep learning, of each connection coefficient (weight) of a multi-layer neural network including an input layer, a hidden layer, and an output layer.

At the time of creating the estimator CLS mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLS is created by performing machine learning using, as the training data, information including a relationship between the endoscopic image information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLS mentioned above, by inputting, as input data, a plurality of curvatures included in the insertion state information in the examination state information generated by the information acquisition unit 271 and a value corresponding to the analysis information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLS mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLS that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for generating, as information indicating an examination state of one endoscopic examination, examination state information including the insertion state information generated based on a plurality of three-dimensional coordinate values included in the insertion position information outputted from the insertion shape observation apparatus 30, and gas-feeding information (gas-feeding information for estimation) indicating a detection result obtained by detecting an operation state of the gas-feeding unit 220. Note that it suffices if the gas-feeding information mentioned above includes information indicating whether gas is supplied to the gas-feeding channel 120 by the gas-feeding unit 220 for a predetermined time period or longer.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLT that is created through learning, by a learning method such as deep learning, of each connection coefficient (weight) of a multi-layer neural network including an input layer, a hidden layer, and an output layer.

At the time of creating the estimator CLT mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLT is created by performing machine learning using, as the training data, information including a relationship between gas-feeding information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLT mentioned above, by inputting, as input data, a plurality of curvatures included in the insertion state information in the examination state information generated by the information acquisition unit 271 and a value corresponding to the gas-feeding information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLT mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLT that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for generating, as information indicating an examination state of one endoscopic examination, examination state information including the insertion state information generated based on a plurality of three-dimensional coordinate values included in the insertion position information outputted from the insertion shape observation apparatus 30, and rigidity control information (variable rigidity portion operation information for estimation) indicating a detection result obtained by detecting an operation state of the rigidity control unit 230. In other words, the rigidity control information is information about operation of the variable rigidity portion provided in the insertion section 11. Note that it suffices if the rigidity control information mentioned above includes information indicating a set value of the degree of rigidity set by the rigidity control unit 230, for example.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLU that is created through learning, by a learning method such as deep learning, of each connection coefficient (weight) of a multi-layer neural network including an input layer, a hidden layer, and an output layer.

At the time of creating the estimator CLU mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLU is created by performing machine learning using, as the training data, information including a relationship between variable rigidity portion operation information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLU mentioned above, by inputting, as input data, a plurality of curvatures included in the insertion state information in the examination state information generated by the information acquisition unit 271 and a value corresponding to the rigidity control information (the set value of the degree of rigidity) in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLU mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLU that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for generating, as information indicating an examination state of one endoscopic examination, examination state information including the insertion state information generated based on a plurality of three-dimensional coordinate values included in the insertion position information outputted from the insertion shape observation apparatus 30, and endoscope information (number-of-uses information for estimation) corresponding to information indicating the number of times of use of the endoscope 10 obtained by detecting information inputted to the input apparatus 50.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLV that is created through learning, by a learning method such as deep learning, of each connection coefficient (weight) of a multi-layer neural network including an input layer, a hidden layer, and an output layer.

At the time of creating the estimator CLV mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLV is created by performing machine learning using, as the training data, information including a relationship between number-of-uses information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLV mentioned above, by inputting, as input data, a plurality of curvatures included in the insertion state information in the examination state information generated by the information acquisition unit 271 and a value corresponding to the endoscope information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLV mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLV that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for generating, as information indicating an examination state of one endoscopic examination, examination state information including the insertion state information generated based on a plurality of three-dimensional coordinate values included in the insertion position information outputted from the insertion shape observation apparatus 30, and insertion section rigidity information (insertion section rigidity information for estimation) indicating rigidity of the insertion section 11. Note that the insertion section rigidity information is information indicating a degree of rigidity that is determined in advance by material, length or the like of the insertion section 11. In other words, the insertion section rigidity information is information indicating a design value that is determined in advance by material, length or the like of the insertion section 11, and is different from the rigidity control information where the degree of rigidity is changed by an operation by a user or an operation state of the rigidity control unit 230.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLY that is created through learning, by a learning method such as deep learning, of each connection coefficient (weight) of a multi-layer neural network including an input layer, a hidden layer, and an output layer.

At the time of creating the estimator CLY mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLY is created by performing machine learning using, as the training data, information including a relationship between insertion section rigidity information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLY mentioned above, by inputting, as input data, a plurality of curvatures included in the insertion state information in the examination state information generated by the information acquisition unit 271 and a value corresponding to the insertion section rigidity information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLY mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLY that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may perform a process for generating, as information indicating an examination state of one endoscopic examination, examination state information including the insertion state information generated based on a plurality of three-dimensional coordinate values included in the insertion position information outputted from the insertion shape observation apparatus 30, and insertion length information (insertion length information for estimation) indicating an insertion length of the insertion section 11 inserted into the subject. Note that the insertion length information is acquired by the insertion shape observation apparatus 30, and is inputted to the information acquisition unit 271 of the system control unit 270. Furthermore, the insertion length information is, in other words, information indicating a part in the intestinal tract (such as sigmoid colon or splenic flexure) where the distal end portion 12 of the insertion section 11 is.

In the case as described above, the pain estimation processing unit 272 may be configured to obtain the estimation result that estimates one pain level among the predetermined plurality of pain levels to be the degree of pain of the subject corresponding to each piece of information included in the examination state information generated by the information acquisition unit 271, by performing processing using an estimator CLX that is created through learning, by a learning method such as deep learning, of each connection coefficient (weight) of a multi-layer neural network including an input layer, a hidden layer, and an output layer.

At the time of creating the estimator CLX mentioned above, for example, machine learning is performed using training data including examination state information similar to the examination state information generated by the information acquisition unit 271 and a label indicating a classification result of classifying the degree of pain of a subject corresponding to the examination state information into one pain level among the predetermined plurality of pain levels. In other words, the estimator CLX is created by performing machine learning using, as the training data, information including a relationship between insertion length information for pre-collection and the pain information for pre-collection, and the pre-collected information including the relationship between the insertion shape information for pre-collection and the pain information for pre-collection, for example. Furthermore, each of the predetermined plurality of pain levels is set as a level among multiple stages including great, small and zero, for example. Moreover, at the time of creating the training data as mentioned above, a task of assigning, to the examination state information, a label according to an evaluation result of evaluating the degree of pain according to the subjective evaluation criterion of the subject taking the endoscopic examination or the objective evaluation criterion of a person other than the subject, such as an expert, is performed.

Accordingly, with the estimator CLX mentioned above, by inputting, as input data, a plurality of curvatures included in the insertion state information in the examination state information generated by the information acquisition unit 271 and a value corresponding to the insertion length information in the examination state information to the input layer of the neural network, a plurality of likelihoods corresponding to respective levels that may be estimated to be the pain level of the subject corresponding to the examination state information may be acquired as output data outputted from the output layer of the neural network, for example. Furthermore, with processing using the estimator CLX mentioned above, one pain level corresponding to a highest likelihood, among the plurality of likelihoods included in the output data outputted from the output layer of the neural network, may be obtained as the estimation result of the pain level of the subject, for example.

In other words, with the processing described above, by performing processing by applying the examination state information, generated by the information acquisition unit 271 in one endoscopic examination, to the estimator CLX that is created by machine learning using information similar to the examination state information, obtained prior to the one endoscopic examination, and that is created as an estimation model different from the estimator CLP, the pain estimation processing unit 272 obtains the estimation result estimating one pain level, among the predetermined plurality of pain levels, to be the degree of pain of the subject in the one endoscopic examination.

According to the present embodiment, for example, the information acquisition unit 271 may be configured to perform a process for generating insertion state information including a plurality of curvatures acquired based on the insertion position information outputted from the insertion shape observation apparatus 30 and the time series data acquired based on the operation force amount information outputted from the operation force amount measurement apparatus 40. In other words, in the present embodiment, the information acquisition unit 271 may be configured to perform a process for generating, as information indicating an insertion state of the insertion section 11 inserted inside the body of a subject taking one endoscopic examination, the insertion state information including at least one of information about the insertion shape of the insertion section 11 or information obtained according to the amount of force applied to the insertion section 11 in the one endoscopic examination. Furthermore, in the case as described above, for example, the pain estimation processing unit 272 may be configured to perform a process for estimating the pain level of the subject and obtaining the estimation result, based on the output data that is obtained by inputting the plurality of curvatures included in the insertion state information generated by the information acquisition unit 271 to the estimator CLP and the output data that is obtained by inputting the time series data included in the insertion state information to the estimator CLW.

According to the present embodiment, the pain estimation processing unit 272 is not limited to outputting the pain level information generated according to the estimation result of the pain level of the subject to the display control unit 260, and, for example, the pain level information may alternatively be outputted to a speaker, not shown. In such a case, for example, a warning sound or voice that is different according to the pain level information generated by the pain estimation processing unit 272 may be outputted from the speaker.

According to the present embodiment, the pain estimation processing unit 272 is not limited to outputting the pain level information generated according to the estimation result of the pain level of the subject to the display control unit 260, and, for example, the pain level information may alternatively be outputted to a lamp, not shown. In such a case, for example, the lamp may be caused to emit light at a blinking interval that is different according to the pain level information generated by the pain estimation processing unit 272.

According to the present embodiment, the pain estimation processing unit 272 is not limited to generating, and outputting to the display control unit 260, the pain level information indicating the estimation result estimating one pain level, among the predetermined plurality of pain levels, to be the degree of pain of the subject, and, for example, the pain estimation processing unit 272 may further generate operation guide information for guiding the insertion operation of the insertion section 11 by the user, according to the estimation result, and may output the operation guide information to the display control unit 260.

More specifically, for example, in the case where the pain level PH is obtained as the estimation result of the pain level of the subject, the pain estimation processing unit 272 generates operation guide information urging that the insertion section 11 be temporarily stopped, and outputs the operation guide information to the display control unit 260. For example, in the case where the pain level PL is obtained as the estimation result of the pain level of the subject, the pain estimation processing unit 272 generates operation guide information urging that an insertion speed, an amount of insertion force and the like of the insertion section 11 be adjusted, and outputs the operation guide information to the display control unit 260. For example, in the case where the pain level PN is obtained as the estimation result of the pain level of the subject, the pain estimation processing unit 272 generates operation guide information urging that the insertion speed, the amount of insertion force and the like of the insertion section 11 be maintained, and outputs the operation guide information to the display control unit 260. For example, in the case where a pain level PO is obtained as the estimation result of the pain level of the subject, the pain estimation processing unit 272 generates operation guide information urging an insertion operation different from a current insertion operation of the insertion section 11 by the user to be performed such that the pain level changes to a pain level that indicates a smaller pain than the pain level PO, and outputs the operation guide information to the display control unit 260.

According to the present embodiment, the pain estimation processing unit 272 is not limited to outputting the operation guide information generated according to the estimation result of the pain level of the subject to the display control unit 260, and, for example, the operation guide information may alternatively be outputted to a speaker, not shown. In such a case, for example, voice urging an operation according to the operation guide information generated by the pain estimation processing unit 272 may be outputted from the speaker.

According to the present embodiment, the pain estimation processing unit 272 is not limited to outputting the operation guide information generated according to the estimation result of the pain level of the subject to the display control unit 260, and, for example, the operation guide information may alternatively be outputted to a lamp, not shown. In such a case, for example, the lamp may be caused to emit light in a lighting state for urging an operation according to the operation guide information generated by the pain estimation processing unit 272.

According to the present embodiment, the pain estimation processing unit 272 is not limited to outputting the pain level information and the operation guide information generated according to the estimation result of the pain level of the subject to the display control unit 260, and, the pain level information and the operation guide information may alternatively be used to control an automatic insertion apparatus that is configured to automatically perform an insertion operation of the insertion section 11. In such a case, for example, in the case where a pain level PP is obtained as the estimation result of the pain level of the subject, operation guide information urging an insertion operation different from a current insertion operation of the insertion section 11 by the automatic insertion apparatus to be performed such that the pain level changes to a pain level that indicates a smaller pain than the pain level PP is generated, and the operation guide information is outputted to the automatic insertion apparatus.

According to the present embodiment, the pain estimation processing unit 272 is not limited to performing processing related to estimation of the pain level of the subject using an estimation model created by machine learning, and may perform processing related to estimation of the pain level of the subject using an estimation model expressed by a polynomial, for example. An example of such a case will be described below.

For example, the pain estimation processing unit 272 calculates a pain value Pa by applying a plurality of curvatures included in the insertion state information generated by the information acquisition unit 271 to an estimation model expressed by a polynomial such as Equation (1) below, and acquires an estimation result estimating the pain level of the subject according to a size of the pain value Pa that is calculated. Note that in Equation (1) below, A₁, A₂, A₃, . . . , Aₛ, Aₛ₊₁ represent approximation parameters, and X₁, X₂, X₃, . . . , Xₛ represent s curvatures included in the insertion state information generated by the information acquisition unit 271.

Pa=A₁X₁+A₂X₂+A₃X₃+. . . AₛXₛ+Aₛ₊₁ (1)

The approximation parameters A₁, A₂, A₃, . . . , Aₛ, Aₛ₊₁ in Equation (1) may be calculated by performing calculation of a determinant indicated by Equation (2) below, for example. Note that in Equation (2), P1, P2, P3, . . . , Pm represent m known pain values corresponding to values obtained by evaluating the degree of pain according to the subjective evaluation criterion of the subject taking the endoscopic examination or the objective evaluation criterion of a person other than the subject, such as an expert. Furthermore, in Equation (2) below, X₁ₘ, X₂ₘ, X₃ₘ, . . . , Xₛₘ represent s known curvatures acquired according to the pain value Pm.

$$( \begin{matrix}P 1 \\ P 2 \\ P 3 \\ ⋮ \\ Pm\end{matrix} ) = ( \begin{matrix}{A}_{1} & {A}_{2} & {A}_{3} & … & {A}_{s} & {A}_{s + 1}\end{matrix} ) ( \begin{matrix}{X}_{11} & {X}_{12} & … & {X}_{1 m} \\ {X}_{21} & {X}_{22} & … & {X}_{2 m} \\ {X}_{31} & {X}_{32} & … & {X}_{3 m} \\ ⋮ & ⋮ & ⋮ & ⋮ \\ {X}_{s 1} & {X}_{s 2} & … & {X}_{sm} \\ 1 & 1 & … & 1\end{matrix} ) \qquad ( 2 )$$

In other words, according to the example described above, the pain estimation processing unit 272 is configured to obtain the estimation result estimating one pain level, among the predetermined plurality of pain levels, to be the degree of pain of the subject in one endoscopic examination, by performing processing by applying the insertion state information, generated by the information acquisition unit 271 in the one endoscopic examination, to the polynomial of (1) described above corresponding to an estimation model that is created using information similar to the insertion state information, obtained prior to the one endoscopic examination.

The estimation model expressed by a polynomial as described above may obtain same effects as the estimation model created by machine learning.

Note that the estimation model to be used by processing by the pain estimation processing unit 272 is not limited to be created as a first-order polynomial such as Equation (1), but may also be created as a second or higher-order polynomial to which a plurality of curvatures included in the insertion state information generated by the information acquisition unit 271 may be applied, for example.

Furthermore, the estimation model to be used by processing by the pain estimation processing unit 272 is not limited to be created as the polynomial such as Equation (1), but may also be created as a polynomial to which both a plurality of curvatures included in the insertion state information in the examination state information generated by the information acquisition unit 271 and the value corresponding to the subject information in the examination state information may be applied.

According to the present embodiment, the pain estimation processing unit 272 is not limited to performing processing related to estimation of the pain level of the subject using an estimation model created by machine learning, but may also perform processing related to estimation of the pain level of the subject using an estimation model that is acquired using a statistical method, for example. An example of such a case will be described below. Note that processing related to creation of the estimation model described below is not limited to be performed by the pain estimation processing unit 272, and may also be performed by an apparatus, such as a computer, different from the main body apparatus 20.

For example, the pain estimation processing unit 272 creates a matrix C by arranging q (q≥2) curvatures corresponding, respectively, to p (p≥2) pain values corresponding to values obtained by evaluating the degree of pain based on the subjective evaluation criterion of the subject taking the endoscopic examination or the objective evaluation criterion of a person other than the subject, such as an expert, and applies singular value decomposition as indicated by Equation (3) below to the matrix C that is generated. Note that in Equation (3) below, V represents a left singular vector, S represents a singular value matrix, and U^{T} represents a transpose matrix of a right singular vector.

C=VSU^{T} (3)

The pain estimation processing unit 272 acquires, as a first component Vx, a greatest value among q components of the q-by-1 left singular vector V obtained by performing singular value decomposition expressed by Equation (3) above, and acquires, as a second component Vy, a second greatest value among elements included in the left singular vector V. In other words, the first component Vx is acquired as a component that is estimated to be most influential in the evaluation of each of p pain values. The second component Vy is acquired as a component that is estimated to be second most influential in the evaluation of each of p pain values.

The pain estimation processing unit 272 acquires each of a curvature Cx corresponding to the first component Vx and a curvature Cy corresponding to the second component Vy from q curvatures corresponding to one pain value Px among the p pain values. In other words, the curvature Cx and the curvature Cy may be expressed as coordinate values (Cx, Cy) on a two-dimensional coordinate system defined by the first component Vx and the second component Vy.

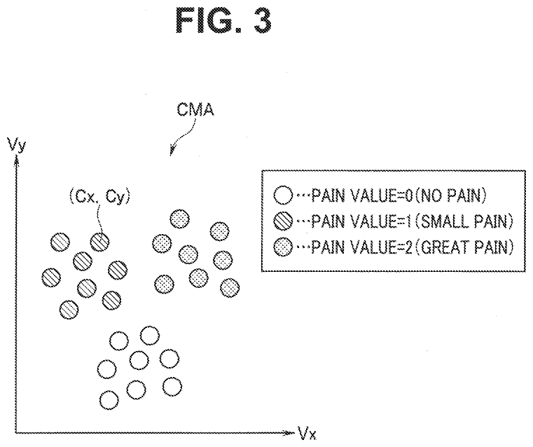

The pain estimation processing unit 272 creates an estimation model CMA as shown in FIG. 3, for example, by performing, in relation to each of the p pain values, a process for acquiring the coordinate values (Cx, Cy) corresponding to the pain value Px. FIG. 3 is a schematic diagram showing an example of an estimation model used in processing by the pain estimation apparatus according to the embodiment.

The pain estimation processing unit 272 acquires the estimation result of the pain level of the subject by acquiring two curvatures corresponding to the first component Vx and the second component Vy from a plurality of curvatures included in the insertion state information in the examination state information generated by the information acquisition unit 271, and by performing a clustering process by a k-nearest neighbor algorithm in a state where the two curvatures (coordinate values) that are acquired are applied to the estimation model CMA.

In other words, according to the example described above, the pain estimation processing unit 272 is configured to obtain the estimation result estimating one pain level, among the predetermined plurality of pain levels, to be the degree of pain of the subject in one endoscopic examination, by performing processing by applying the insertion state information, generated by the information acquisition unit 271 in the one endoscopic examination, to the estimation model CMA created using information similar to the insertion state information, obtained prior to the one endoscopic examination.

The estimation model acquired using the statistical method as described above may obtain same effects as the estimation model created by machine learning.

Note that the present invention is not limited to the embodiment described above, and various modifications, combinations and applications are naturally possible within the scope not departing from the spirit of the invention.

What is claimed is:

1. A pain estimation apparatus comprising:
an information acquisition unit configured to perform a process for acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and
pain estimation processing means configured to generate pain information about pain of the subject based on the examination state information for estimation including the information different from either of the insertion shape information for estimation and the operation force amount information for estimation, and the insertion state information for estimation.

2. The pain estimation apparatus according to claim 1, wherein the information acquisition unit is configured to perform a process of calculating a plurality of curvatures at a plurality of positions, respectively, in the insertion section, as a process for acquiring the insertion shape information for estimation.

3. The pain estimation apparatus according to claim 1, wherein the information acquisition unit is configured to perform a process of generating an insertion shape image showing the insertion shape of the insertion section, as a process for acquiring the insertion shape information for estimation.

4. The pain estimation apparatus according to claim 1, wherein the information acquisition unit is configured to perform a process for acquiring, as information indicating an examination state of the endoscopic examination, examination state information for estimation including the insertion state information for estimation and subject information for estimation about the subject, and
the pain estimation processing means generates, based on the examination state information for estimation, the pain information corresponding to the examination state information for estimation including the insertion state information for estimation and the subject information for estimation.

5. The pain estimation apparatus according to claim 1, wherein
the information acquisition unit is configured to perform a process for acquiring, as information indicating an examination state of the endoscopic examination, examination state information for estimation including the insertion state information for estimation and endoscopic image information for estimation about an endoscopic image obtained by performing image pickup of inside of the body of the subject by the endoscope, and
the pain estimation processing means generates, based on the examination state information for estimation, the pain information corresponding to the examination state information for estimation including the insertion state information for estimation and the endoscopic image information for estimation.

6. The pain estimation apparatus according to claim 1, wherein
the information acquisition unit acquires, as information indicating an examination state of the endoscopic examination, examination state information for estimation including the insertion state information for estimation and gas-feeding information for estimation about an operation state of a gas-feeding unit configured to perform an operation for supplying gas to the endoscope, and
the pain estimation processing means generates, based on the examination state information for estimation, the pain information corresponding to the examination state information for estimation including the insertion state information for estimation and the gas-feeding information for estimation.

7. The pain estimation apparatus according to claim 1, wherein
the information acquisition unit acquires, as information indicating an examination state of the endoscopic examination, examination state information for estimation including the insertion state information for estimation and variable rigidity portion operation information for estimation about an operation of a variable rigidity portion provided in the insertion section, and
the pain estimation processing means generates, based on the examination state information for estimation, the pain information corresponding to the examination state information for estimation including the insertion state information for estimation and the variable rigidity portion operation information for estimation.

8. The pain estimation apparatus according to claim 1, wherein
the information acquisition unit acquires, as information indicating an examination state of the endoscopic examination, examination state information for estimation including the insertion state information for estimation and number-of-uses information for estimation about a number of times of use of the endoscope, and
the pain estimation processing means generates, based on the examination state information for estimation, the pain information corresponding to the examination state information for estimation including the insertion state information for estimation and the number-of-uses information for estimation.

9. The pain estimation apparatus according to claim 1, wherein
the information acquisition unit acquires, as information indicating an examination state of the endoscopic examination, examination state information for estimation including the insertion state information for estimation and insertion section rigidity information for estimation about rigidity of the insertion section, and
the pain estimation processing means generates, based on the examination state information for estimation, the pain information corresponding to the examination state information for estimation including the insertion state information for estimation and the insertion section rigidity information for estimation.

10. The pain estimation apparatus according to claim 1, wherein
the information acquisition unit acquires, as information indicating an examination state of the endoscopic examination, examination state information for estimation including the insertion state information for estimation and insertion length information for estimation about an insertion length of the insertion section inside the subject, and
the pain estimation processing means generates, based on the examination state information for estimation, the pain information corresponding to the examination state information for estimation including the insertion state information for estimation and the insertion length information for estimation.

11. The pain estimation apparatus according to claim 1, wherein the pain estimation processing means further generates operation guide information for guiding an insertion operation of the insertion section, according to the pain information that is generated.

12. A pain estimation method comprising:
acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and
generating pain information about pain of the subject based on the examination state information for estimation including the information different from either of the insertion shape information for estimation and the operation force amount information for estimation, and the insertion state information for estimation.

13. A non-transitory recording medium recording a program, the program causing a computer to:
perform a process for acquiring, in an endoscopic examination, examination state information for estimation which includes insertion state information for estimation including at least one of insertion shape information for estimation about an insertion shape of an insertion section of an endoscope inserted inside a body of a subject or operation force amount information for estimation about an amount of force applied to the insertion section in the endoscopic examination, and information different from either of the insertion shape information for estimation and the operation force amount information for estimation; and
perform a process for generating pain information about pain of the subject based on the examination state information for estimation including the information different from either of the insertion shape information for estimation and the operation force amount information for estimation, and the insertion state information for estimation.
